(19) United States
(12) Patent Application Publication
(10) Pub. No.: US 2005/0113907 A1
(43) Pub. Date: May 26, 2005

(54) Method and apparatus for treating vulnerable coronary plaques using drug-eluting stents

(76) Inventor: David R. Fischell, Fair Haven, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/969,260

(22) Filed: Oct. 20, 2004

Related U.S. Application Data
(62) Division of application No. 10/185,021, filed on Jun. 28, 2002.

(51) Int. Cl. A61F 2/06

(52) U.S. Cl. 623/1.42

(57) ABSTRACT

A drug-eluting intravascular stent comprising an anti-restenosis agent covered by a biodegradable coating, and a method for treating vulnerable plaque in coronary vessels using said stent is disclosed. The biodegradable layer covers at least a portion of the drug-eluting layer of the stent, and is adapted to slowly erode over a preset period of time, preventing the release of therapeutic amounts of the anti-restenosis agent from the drug-eluting layer during the preset period. By delaying the release of the anti-restenosis agent, a thin layer of neointima will grow during the preset period. This tissue growth is sufficient to encapsulate the stent and cover the vulnerable plaque, but not significant enough to cause harmful restenosis or occlusion of the vessel. Once the biodegradable coating is eroded, the anti-restenosis agent begins release from the drug-eluting layer, and the progression of neointimal hyperplasia ceases.

### CROSS REFERENCE TO RELATED APPLICATION

The present application is a divisional of U.S. patent application Ser. No. 10/185,021 filed on Jun. 28, 2002, and claims priority thereto under 35 U.S.C. § 121.

### FIELD OF USE

This invention relates generally to improved medical apparatus and methods for treating vascular tissues, and more particularly to improved drug-eluting intravascular stents, and the use of the improved intravascular stents for treating vulnerable plaques.

### BACKGROUND OF THE INVENTION

Cardiovascular disease is one of the leading causes of death worldwide. Traditionally, cardiovascular disease was thought to originate from severe blockages created by atherosclerosis, the progressive accumulation of non-vulnerable plaque in the coronary arteries. This constriction or narrowing of the affected vessel could ultimately lead to angina, and eventually coronary occlusion, sudden cardiac death, and/or thrombotic stroke.

Traditional atherosclerosis therapies consist of balloon angioplasty and stenting. While it has been shown that intravascular stents are an excellent means to maintain the patency of blood vessels following balloon angioplasty, neointima and/or intimal hyperplasia through the openings of the expanded stent meshes as a result of tissue injury remained a major cause for stent restenosis.

Drug coated stents, such as the CYPHER™ sirolimus eluting stent by Cordis, a Johnson & Johnson Company, have been shown to virtually eliminate injury related tissue growth inside the stent that can cause restenosis. Sirolimus, in fact, works so well that there is essentially no neointimal hyperplasia (tissue growth) inside the stent.

Recent studies have lead to a shift in understanding of atherosclerosis and uncovered another major vascular problem not yet well treated. Scientists theorize that at least some coronary disease is an inflammatory process, in which inflammation causes plaque to rupture. This inflamed plaque is known as atherosclerotic vulnerable plaque.

Vulnerable plaque consists of a lipid-rich core covered by a thin layer of inflammatory cells. These plaques are prone to rupture and erosion, and can cause significant infarcts if the thin inflammatory cell layer ruptures or ulcerates. When the inflammatory cells erode or rupture, the lipid pool is exposed to the blood flow, forming clots in the artery. These clots may grow rapidly and block the artery, or detach and travel downstream, leading to thromboembolic events, unstable angina, myocardial infarction, and/or sudden death. In fact, some recent studies have suggested that plaque rupture may trigger 60 to 70% of all fatal myocardial infarctions. See U.S. Pat. No. 5,924,997 issued to Campbell and U.S. Pat. No. 6,245,026 issued to Campbell et al. for further descriptions of vulnerable plaques.

Early methods used to detect atherosclerosis lacked the diagnostic tools to visualize and identify vulnerable plaque in cardiac patients. However, new diagnostic technologies are under development to identify the location of vulnerable plaques in the coronary arteries. These new devices include refined magnetic resonance imaging (MRI), thermal sensors that measure the temperature of the arterial wall on the premise that the inflammatory process generates heat, elasticity sensors, intravascular ultrasound, optical coherence tomography (OCT), contrast agents, and near-infrared and infrared light. What is not currently clear, however, is how to treat these vulnerable plaque locations once they are found.

Treating vulnerable plaque by using balloon angioplasty followed by traditional stenting would provide less than satisfactory results. Balloon angioplasty by itself may rupture the vulnerable plaque exposing the underlying fresh tissue cells (collagen or damaged endothelium) to the blood flow. This condition ultimately leads to the formation of a blood clot that may partially or completely occlude the vessel. In addition, while bare (uncoated) stents will induce neointimal hyperplasia that will provide a protective cover over the vulnerable plaque, restenosis remains a major problem that may create more risk to the patient than the original vulnerable plaque.

Drug-eluting stents presently known in the art, such as sirolimus coated stents, prevent restenosis and do not allow neointimal hyperplasia, thus prohibiting and/or preventing tissue growth that may cover and seal the vulnerable plaque, allowing the potential for a rupture at a later time.

What is needed is an apparatus and method for treating vulnerable plaque by sealing and/or covering the inflammatory cells to prevent erosion or rupture in the future without having the additional risk of restenosis.

### SUMMARY OF THE INVENTION

It is an object of this invention to have an anti-restenosis drug-eluting stent with a thin biodegradable layer coated over the stent to delay release of the anti-restenosis agent.

Another object of this invention is to have an anti-thrombogenic agent embedded in the thin biodegradable layer.

Still another object of this invention is to have an anti-platelet agent embedded in the thin biodegradable layer.

It is a further object of this invention to have a method for treating vulnerable plaque comprising first detection of a vulnerable plaque followed by implantation of an improved drug-eluting stent.

The present invention is for a medical apparatus for treating vulnerable plaque in a vessel. The medical apparatus comprises an intravascular stent having a tubular configuration of structural members, the tubular configuration having proximal and distal open ends, and defining a longitudinal axis therebetween. A drug-eluting layer containing an anti-restenosis agent covers at least a portion of the intravascular stent structural members. A biodegradable layer covers at least a portion of the drug-eluting layer, and is adapted to slowly erode over a preset period of time. The biodegradable layer is also adapted to prevent release of the anti-restenosis agent from the drug-eluting layer during the preset period of time. In a preferred embodiment, the anti-restenosis agent comprises sirolimus, including any/all analogs thereof. The drug-eluting layer may further comprise a lipid lowering agent or statin, singly or in combination thereof.

The present invention further includes a method for treating vulnerable plaque in a vessel. The steps comprising the method include first identifying the location of the vulnerable plaque in the vessel. A drug-eluting intravascular stent having a tubular configuration of structural members is delivered to the site of the vulnerable plaque. The intravascular stent comprises a drug-eluting layer containing an anti-restenosis agent coated over at least a portion of the intravascular stent structural members. A biodegradable layer adapted to slowly erode over a preset period of time covers at least a portion of the drug-eluting layer. The biodegradable layer is also adapted to prevent release of therapeutic amounts of the anti-restenosis agent from the drug-eluting layer during the preset period of time. As used herein, the term “therapeutic amount” refers to an amount of anit-restenosis agent that can limit or prevent neointimal hyperplasia. The intravascular stent is deployed into the wall of the vessel over the area of the vulnerable plaque. The anti-restenosis agent is then caused to be released from the drug-eluting layer.

The present invention further contemplates a system and method for correcting undersized stents by allowing limited tissue growth to anchor the deployed device.

### BRIEF DESCRIPTION OF THE DRAWINGS

FIG. 1A is a perspective view of an exemplary stent in the expanded state.

FIG. 1B is an enlarged view of a section of the stent illustrated in FIG. 1A.

FIG. 2A is transverse cross section of a strut from a drug-eluting stent as is known in the prior art.

FIG. 2B is an alternate embodiment of a strut from a drug-eluting stent as is known in the prior art.

FIG. 3 illustrates a partial cross-sectional view showing the anatomy of a typical coronary vessel with some vascular disease.

FIG. 4 illustrates an intravascular stents disposed within a coronary vessel with some vascular disease to maintain the patency of the vessel.

FIG. 5A is a transverse cross section of a strut from a drug-eluting stent having a thin biodegradable layer designed to delay the release of the agent from the drug-eluting layer according to one embodiment of the present invention.

FIG. 5B is a transverse cross section of a strut from a drug-eluting stent over-coated by a slow-release layer and a thin biodegradable layer designed to delay the release of the agent from the slow release layer according to one embodiment of the present invention.

FIG. 6 is a partial cross sectional view of a coronary vessel illustrating the thin layer of neointima encapsulating the intravascular stent disposed within the vessel according to one embodiment of the present invention.

### DETAILED DESCRIPTION OF THE DRAWINGS

The present invention discloses a stent-based apparatus for treating vulnerable plaque comprising an intravascular drug-eluting stent, wherein one or more structural elements of the stent are coated with a thin biodegradable layer designed to delay the release of the agent from the drug-eluting layer.

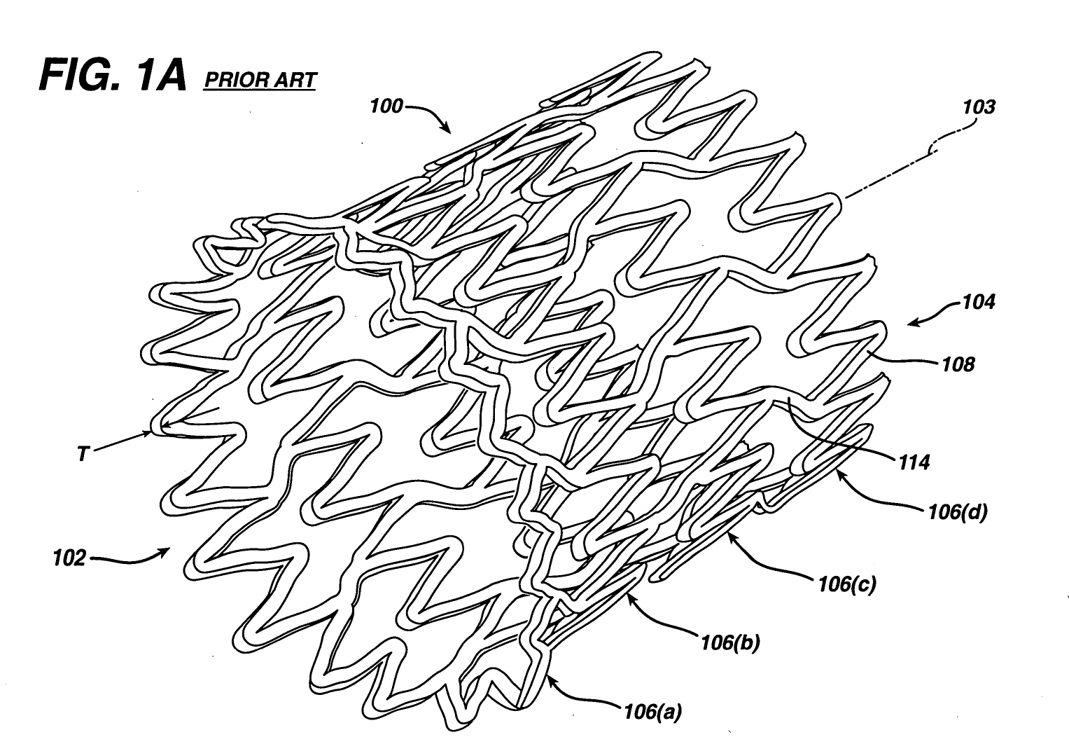

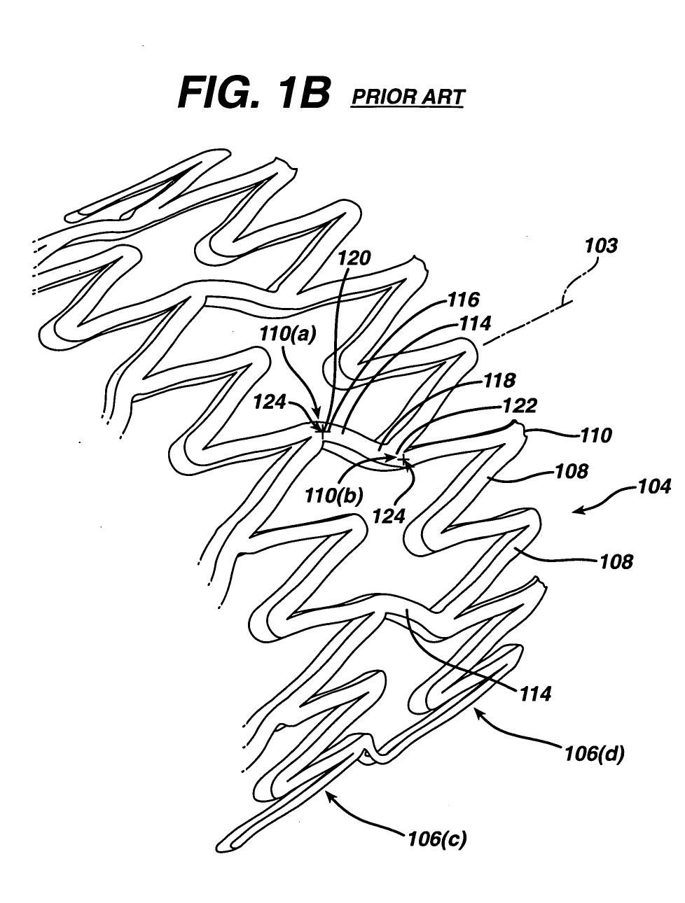

Perspective views of a typical stent in the expanded state are shown in FIGS. 1A and 1B. Although a Z or S shaped pattern stent is shown for the purpose of example, the illustration is not to be construed as limiting the scope of the invention.

A stent 100 comprises a tubular configuration of structural elements having proximal and distal open ends 102, 104 and defining a longitudinal axis 103 extending therebetween. The stent 100 has a first diameter (not shown) for insertion into a patient and navigation through the vessels, and a second diameter for deployment into the target area of a vessel, with the second diameter being greater than the first diameter. The stent 100 may be either a balloon expandable stent or self-expanding stent.

The stent 100 structure comprises a plurality of adjacent hoops 106(a)-(d) extending between the proximal and distal ends 102, 104. The hoops 106(a)-(d) include a plurality of longitudinally arranged strut members 108 and a plurality of loop members 110 connecting adjacent struts 108. Adjacent struts 108 are connected at opposite ends in a substantially S or Z shaped pattern so as to form a plurality of cells. However, one of ordinary skill in the art would recognize that the pattern shaped by the struts is not a limiting factor in this invention, and other shaped patterns may be used. The plurality of loops 110 have a substantially semi-circular configuration and are substantially symmetric about their centers.

The stent 100 structure further comprises a plurality of bridge members 114, which connect adjacent hoops 106(a)-(d). Each bridge comprises two ends 116, 118. One end of each bridge 114 is attached to one loop 110 on one hoop, for examples hoop 106(c), and the other end of each bridge 114 is attached to one loop 110 on an adjacent hoop, for example hoop 106(d). The bridges 114 connect adjacent hoops 106(a)-(d) together at bridge to loop connection regions 120, 122. By way of example, bridge end 116 is connected to loop 110(a) at bridge to loop connection regions 120, and bridge end 118 is connected to loop 110(b) at bridge to loop connection region 122. Each bridge to loop connection region includes a center 124. The bridge to loop connection regions 120, 122, are separated angularly with respect to the longitudinal axis 103 of the stent 100.

To increase the effectiveness of intravascular stents and reduce restenosis caused by neointima and/or intimal hyperplasia (neointimal hyperplasia), many stents today are coated with a drug-eluting layer that retards tissue growth. One such anti-restenosis (anti-proliferate) agent comprises sirolimus in combination with other agents. For the purpose of this application, the term drug-eluting layer includes but is not limited to cytostatic anti-restenosis agents, such as agents comprising sirolimus.

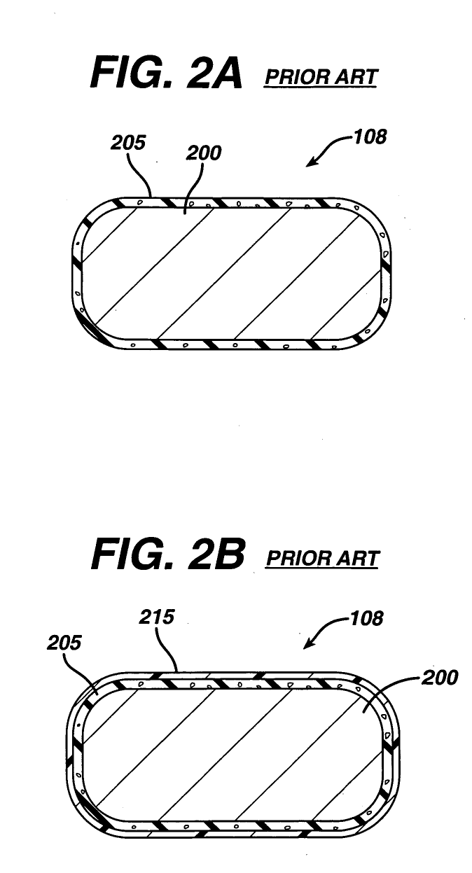

A transverse cross section of the strut 108 from a typical drug-eluting stent, as is well known in the art, is illustrated in FIGS. 2A and 2B. In each embodiment, the stent strut 108 comprises a strut core 200 coated by one or more layers. The strut cores 200 in the prior art stents are typically comprised of a metallic material, such as stainless steel, tantalum or nitinol.

Turning to FIG. 2A, the stent strut 108 comprises a metallic strut core 200 coated by a drug-eluting layer 205. A described earlier, the drug-eluting layer comprises an agent that minimizes restenosis caused by neointima and/or intimal hyperplasia through the openings of the expanded stent mesh. Such stents are currently being used with agents such as paclitaxel and Actinamycin D, which have been shown effective in reducing restenosis in early pilot studies.

FIG. 2B illustrates an alternate embodiment of the prior art drug-eluting stent strut 108. In the embodiment shown, the drug-eluting stent strut 108 comprises a metal strut 200 coated by a drug-eluting layer 205 that further comprises a porous slow release layer 215. The porosity of the slow release layer 215 allows the agent in the drug-eluting layer 205 to permeate at a controlled rate upon stent implantation. This combination has been found to eliminate neointimal hyperplasia that can cause in-stent restenosis. One example of this type of drug-eluting stent currently being used is the Cypher™ sirolimus drug-eluting stent by Cordis, a Johnson and Johnson company.

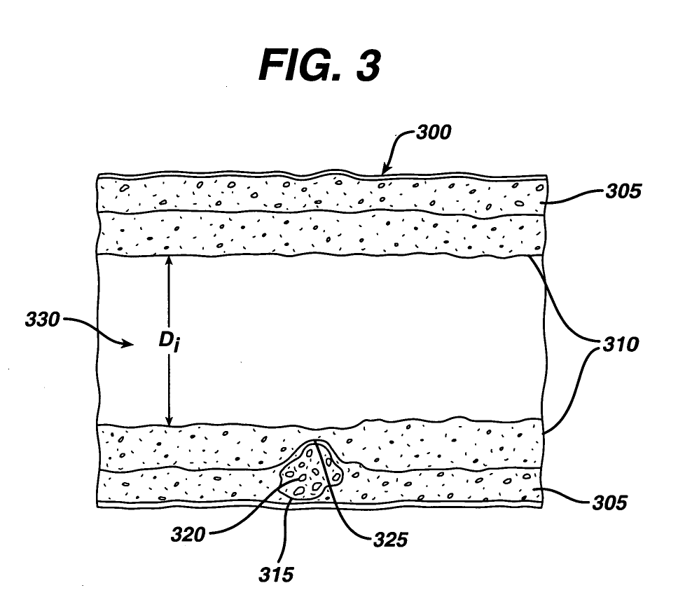

A described earlier, the present invention comprises improved medical apparatus and methods for treating vascular disease, and particularly cardiovascular disease including vulnerable plaques. A partial cross-sectional view showing the anatomy of a typical coronary vessel, artery 300, is shown in FIG. 3. The artery 300 is comprised of arterial walls 305 forming a lumen 330 within the artery 300. Also illustrated in FIG. 3 are non-vulnerable and vulnerable plaques 310, 315 respectively, which represent some vascular diseases that can be treated using the present invention.

The lumen 330 is a tubular chamber formed by the arterial walls and provides a conduit for blood to be carried from the heart through the body. Traditionally, vascular disease, and particularly cardiovascular disease, was thought to originate from severe blockages created by atherosclerosis, or the progressive accumulation of the non-vulnerable plaque 310 formed along the inside surface of the arterial wall 305. As one of ordinary skill in the art would recognize, the accumulation of the non-vulnerable plaque 310 along the interior surface of the arterial walls 305 decreases the internal diameter Di of the lumen 330. This narrowing of the affected artery 300 could ultimately lead to angina, and eventually coronary occlusion, sudden cardiac death, and thrombotic stroke.

Recent studies have identified another major vascular problem that can cause the rapid occlusion of the artery 300—the rupture of the vulnerable plaque 315. Vulnerable plaque may exist in combination with non-vulnerable plaque 310, but it may also exist alone. The vulnerable plaque 315 is comprised of a lipid rich core 320 covered by a thin fibrous cap of inflammatory cells 325. The inflammatory cells 325 are relatively thin and prone to erosion and rupture. As described earlier, if the inflammatory cells 325 ruptures, the lipid pool 320 is exposed to the blood flow, forming clots in the artery 300. These clots can rapidly occlude the artery 300, and may also detach from the arterial wall 305 and travel through the artery 300 precipitating various cardiac events.

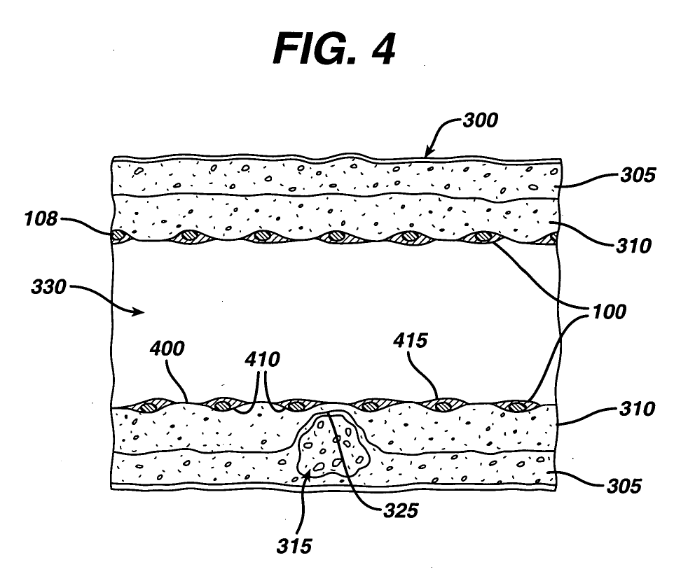

Intravascular stents, similar to stent 100, have been successfully used, both alone and in combination with balloon angioplasty, to maintain the patency of blood vessels partially occluded by non-vulnerable plaque. FIG. 4 illustrates an intravascular stent 100 disposed within the artery 300 exemplifying such use.

For the purpose of illustration, the non-vulnerable plaque 310 depicted in FIG. 4 has been compressed by the balloon angioplasty procedure, and the stent 100 is engaged within the compressed non-vulnerable plaque 310. The correct placement of the stent 100 results in mounds 400 protruding between the struts 108 after the struts 108 have been embedded in the non-vulnerable plaque 310. These tissue mounds 400 retain endothelial cells that can provide for the re-endothelialization of the artery wall. Endothelial regeneration of the artery wall proceeds in a multicentric fashion with the endothelial cells migrating to, and over, the stent struts 108. The satisfactory, rapid endothelialization results in a thin tissue layer 415 encapsulating the stent strut 108.

The struts 108 also form shallow troughs or depressions 410 in the non-vulnerable plaque 310 and the arterial wall 305. These depressions contribute to injury of the artery wall 305, and initiate a thrombotic and inflammatory response, leading to undesirable tissue growth in the form of neointima and/or intimal hyperplasia. If left untreated, this neointima and/or intimal hyperplasia can lead to stent restenosis and partially or completely occlude the artery 300 over time. To counteract the effects of restenosis, prior art stents, such as the sirolimus coated stents illustrated in FIGS. 2A and 2B, utilize anti-restenosis agents to effectively prevent the neointima and/or intimal hyperplasia without inhibiting the endothelial regeneration of cell that anchor the stent 100 in place.

While the prior art intravascular stents shown in FIGS. 2A and 2B may control restenosis, they do little to protect the inflammatory cells 325 from erosion or rupture. One method contemplated by the present invention to protect the inflammatory cells 325 from erosion or rupture is to cover or encapsulate the vulnerable plaque with a thin layer of tissue growth. This tissue growth must be controlled so as to allow the tissue layer to become thick enough to protect the inflammatory cells 325 from erosion and rupture, yet thin enough to minimize occlusion of the artery 300. The tissue growth may also facilitate the anchoring of an undersized stent.

The present invention envisions utilizing an improved drug-eluting stent to control neointimal hyperplasia, while still allowing a thin neointima tissue layer to form over the inflammatory cells 325. In a preferred embodiment, a drug-eluting stent is coated with one or more outer layers that prohibit perfusion of the anti-restenosis agent from the drug-eluting layer for a predetermined period of time. These layers are biodegradable and slowly erode over a period of days or weeks. For the purposes of this application, the time over which the outer layer(s) erode can be called the release delay. When the outer layer is eroded, the anti-restenosis agent release from the drug-eluting layer begins.

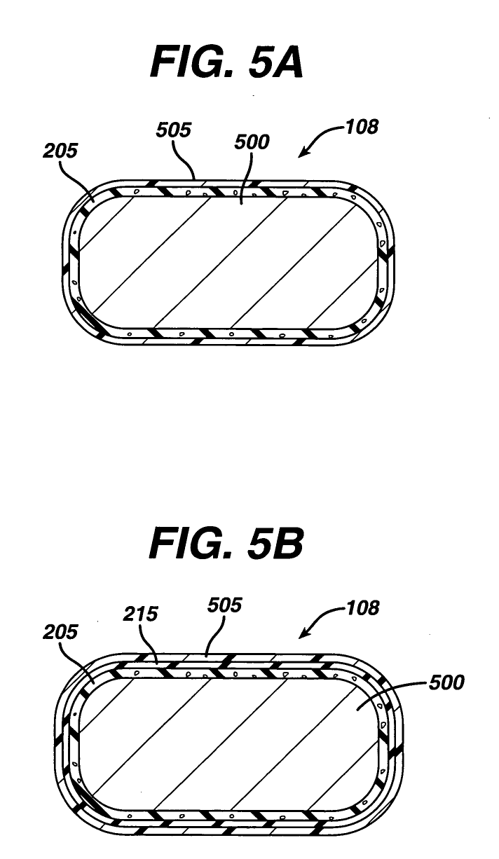

Turning to FIGS. 5A and 5B, there is illustrated transverse cross sectional views of the stent struts 108 for an improved drug-eluting stent according to two embodiments of the present invention. In each embodiment, the stent strut 108 comprises a strut core 500 covered by one or more coatings. In a preferred embodiment of the invention, the strut core 500 is comprised of a metallic material such as stainless steel or tantalum in balloon expandable stents, or Nitinol for self-expanding stents. However any material known in the art to possess characteristics desirable for stent construction may be used.

A drug-eluting layer 205, as is known in the art, covers the strut core 500 illustrated in FIGS. 5A and 5B. The drug-eluting layer 205 comprises an anti-restenosis agent that has been found to minimize and/or prevent restenosis caused by neointima and/or intimal hyperplasia. In a preferred embodiment, the drug-eluting layer 205 comprises an anti-proliferative agent; such as paclitaxel, Alkeran, Cytoxan, Leukeran, Cis-platinum, BiCNU, Adriamycin, Doxorubicin, Cerubidine, Idamycin, Mithracin, Mutamycin, Fluorouracil, Methotrexate, Thoguanine, Toxotere, Etoposide, Vincristine, Irinotecan, Hycamptin, Matulane, Vumon, Hexalin, Hydroxyurea, Gemzar, Oncovin, Etophophos, tacrolimus (FK506), Everolimus, or any of the following analogs of sirolimus: SDZ-RAD, CCI-779, 7epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy, 2-desmethyl and proline.

The drug-eluting layer 205 may also comprise lipid lowering agents and/or statins, singly or in combination thereof, to influence the composition of the lipid pool in the vulnerable plaque. The lipid lowering agents and/or statins may also be contained in a second drug-eluting layer (not shown).

In addition, the drug-eluting layer 205 may also comprise antithrombogenic agents, such as heparin or coumadin, or anti-platelet agents, such as Plavix or ReoPro.

In the embodiment of the invention illustrated in FIG. 5B, the drug-eluting layer 205 additionally comprises a slow release layer 215 which allows the anti-restenosis agent in the drug-eluting layer 205 to slowly permeate into the blood stream. This slow release layer 215 may, for example, comprise polyethylene-co-vinylacetate and/or polybutylmethacrlate.

To obtain the necessary release delay, the improved stent 100 of the present invention comprises a thin biodegradable layer 505 coated over the strut 108. In the embodiment of the invention illustrated in FIG. 5A, the biodegradable layer 505 is designed to delay the release of the anti-restenosis agent from the drug-eluting layer 205 that covers the strut core 500. Similarly, in the embodiment of the invention illustrated in FIG. 5B, the biodegradable layer 505 is designed to delay the slow release of the anti-restenosis agent from the drug-eluting layer 205, through the slow release layer 215. It is also envisioned that the biodegradable layer 505 may greatly reduce the release of therapeutic amounts of the anti-restenosis agent rather than totally prevent release.

This delay in release provides the added benefit of allowing controlled neointima tissue growth before the drug-eluting layer 205 activates and suppresses the neointimal hyperplasia.

The biodegradable layer 505 may comprise a material having a wide range of biodegradation properties, such as, for example a polymer. In a preferred embodiment of the invention, the biodegradable layer 505 comprises polylactide, polyglycolide, copolymer of polyglycolide and polylactide, or poly-ε-caprolactone. In addition, some recently synthesized biodegradable dextran-based (polysaccharide) polymers could also be considered. Antithrombogenic agents, such as heparin or coumadin, or anti-platelet agents, such as Plavix or ReoPro, could be mixed into the thin biodegradable layer 505 to provide additional benefit to the patient. In addition, lipid lowering agents and/or statins, singly or in combination, may be contained in the biodegradable layer 505.

The biodegradable material may be applied to the stent strut 108 by any know means. In one embodiment of the invention, the biodegradable material is put into a solution and sprayed over the strut 108 until the proper thickness is achieved. Alternatively, the stent 100 may be immersed into a bath of liquefied biodegradable material until the proper thickness is achieved. As the biodegradable material dries and solidifies it forms the biodegradable layer 505.

Typically, current drug-eluting stents release the anti-restenosis agent over a two (2) week period. Although this release may be time released and/or slow released, the present invention will delay commencement of therapeutic amounts of the anti-restenosis agent release by the release delay period—typically between 1 day and 4 weeks. The length of the release delay period may be determined by several factors, including the patient's blood chemistry. In a preferred embodiment the release delay period of two (2) weeks should allow sufficient neointima tissue growth.

The thickness of the biodegradable layer 505 necessary to achieve the proper release delay is dependent on the erosion properties of the biodegradable material. In one embodiment of the invention, the material used in the biodegradable layer 505 is an absorbable elastomer based on 45:55 mole percent copolymer of ε-caprolactone and glycolide, with an IV of 1.58 (0.1 g/dl in hexafluoroisopropanol [HFIP] at 25 degrees Celsius) that was dissolved five percent (5%) by weight in acetone and separately fifteen percent (15%) by weight in 1,1,2-trichloroethane. The synthesis of the elastomer is described in U.S. Pat. No. 5,468,253 issued to Bezwada et al., which is herein incorporated by reference.

A stent having a drug eluting layer 205 (with or without a slow release layer 215) over a strut core 500 is dip coated in the five percent (5%) solution until a top coating 505 of approximately 100 micrograms of polymer coating is achieved after air drying at room temperature. Methods for dip coating the stent are known in the art. One such method is disclosed in U.S. Pat. No. 6,153,252 issued to Hossainy et al., which is incorporated herein by reference.

This method will yield a polymer top coating 505 of between 1 and 10 micrometers in thickness. A biodegradable polymer coating of this approximate configuration will provide a release delay period of approximately two (2) weeks before therapeutic amounts of agent are released from the drug-eluting layer 205.

In another embodiment of the invention, the material used in the biodegradable layer 505 is a copolymer based on 40:60 mole percent poly ε-caprolactone-co-L-Lactide). The synthesis of the copolymer is described in U.S. Pat. No. 6,153,252 issued to Hossainy et al, previously incorporated by reference.

As described earlier, a stent having a drug eluting layer 205 (with or without a slow release layer 215) over a strut core 500 is dip coated in the 40:60 mole percent poly (ε-caprolactone-co-L-Lactide) solution until a top coating 505 of approximately 100 micrograms of copolymer coating is achieved.

This method will yield a polymer top coating 505 of between 1 and 10 micrometers in thickness. A biodegradable copolymer coating of this approximate configuration will similarly provide a release delay period of approximately two (2) weeks before therapeutic amounts of agent are released from the drug-eluting layer 205.

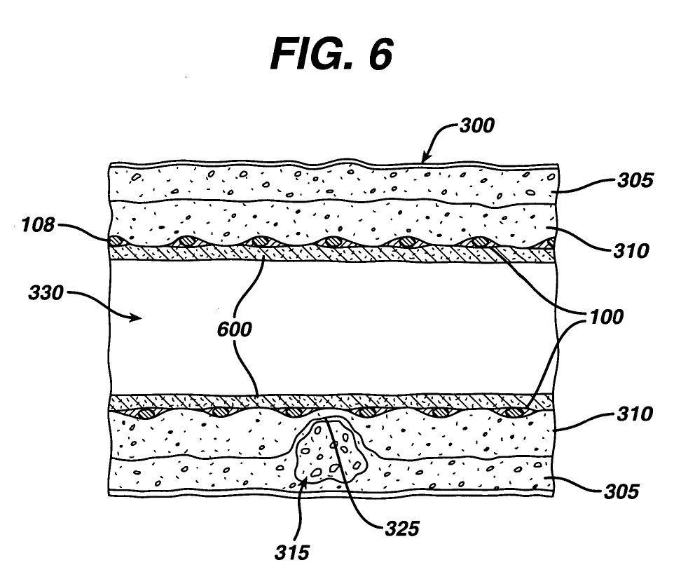

Delaying the release of the anti-restenosis agent from the drug-eluting layer during the release delay period allows a thin layer of neointima tissue to grow. This tissue growth is sufficient to cover or encapsulate the stent, providing a tissue cover over the vulnerable plaque 315, but not significant enough to cause harmful restenosis or occlusion of the artery 300. FIG. 6 is a partial cross sectional view illustrating the thin layer of neointima 600 encapsulating the intravascular stent 100 disposed within the artery 300.

Once the biodegradable layer 505 is eroded, the anti-restenosis agent begins release, and the progression of neointima and/or intimal hyperplasia ceases. The condition of the artery 300 will essentially be “frozen” in time with respect to the neointima tissue growth. The thin layer of the neointima 600 remaining is sufficient to seal over and cover the vulnerable plaque 315, and provide sufficient protection for the inflammatory cells 325 against rupture and erosion.

In operation, a prerequisite step to treating a patient with the improved intravascular stent of the present invention is to detect and locate an area of vulnerable plaque 315. Numerous devices are becoming available to detect the presence of vulnerable plaques. These new devices include refined magnetic resonance imaging (MRI), thermal sensors that measure the temperature of the arterial wall on the premise that the inflammatory process generates heat, elasticity sensors, intravascular ultrasound, optical coherence tomography (OCT), contrast agents, and near-infrared and infrared light.

In addition, in cases where a patient is being treated for another coronary lesion it would be obvious to search for such vulnerable plaques especially in major vessels such as the Left Main, LAD, Circumflex and Right Coronary arteries.

When an area of vulnerable plaque is found the improved drug-eluting stent of the present invention can be delivered to the site of the vulnerable plaque and deployed into the wall of the vessel over the area of vulnerable plaque.

These and other objects and advantages of this invention will become obvious to a person of ordinary skill in this art upon reading of the detailed description of this invention including the associated drawings.

Various other modifications, adaptations, and alternative designs are of course possible in light of the above teachings. Therefore, it should be understood at this time that within the scope of the appended claims the invention might be practiced otherwise than as specifically described herein.

1) A method for treating vulnerable plaque in a vessel, the method comprising the steps of:
a) identifying the location of the vulnerable plaque in the vessel;
b) delivering a drug-eluting intravascular stent comprising a tubular configuration of structural members to the location of the vulnerable plaque, the intravascular stent comprising a drug-eluting layer coated over at least a portion of the intravascular stent structural members, the drug-eluting layer comprising an anti-restenosis agent; and a biodegradable layer covering at least a portion of the drug-eluting layer, the biodegradable layer being adapted to slowly erode over a preset period of time, the biodegradable layer also being adapted to prevent release of therapeutic amounts of the anti-restenosis agent from the drug-eluting layer during the preset period of time;
c) deploying the intravascular stent into the wall of the vessel over the location of the vulnerable plaque; and
d) causing therapeutic amounts of the anti-restenosis agent to elute from the drug-eluting layer into the location of the vulnerable plaque after the preset period of time.

2) The method of claim 1 wherein the anti-restenosis agent comprises sirolimus.

3) The method of claim 1 wherein the biodegradable layer contains an anti-thrombogenic agent.

4) The method of claim 3 wherein the anti-thrombogenic agent is heparin.

5) The method of claim 1 wherein the biodegradable layer contains an anti-platelet agent.

6) The method of claim 5 wherein the anti-platelet agent is ReoPro.

7) The method of claim 1 wherein the drug-eluting layer comprises a lipid lowering agent.

8) The method of claim 1 wherein the drug-eluting layer comprises a statin.

9) The method of claim 1 wherein the biodegradable layer comprises a lipid lowering agent.

10) The method of claim 1 wherein the biodegradable layer comprises a statin.
